(19) United States
(12) Patent Application Publication
(10) Pub. No.: US 2024/0182129 A1
(43) Pub. Date: Jun. 6, 2024

(54) SYSTEM AND METHOD FOR DRIVING MONITORING AND ANALYZING AND GENERATING ALERTS TO USERS IN REAL-TIME

(71) Applicant: ANIRUDHA SURABHI VENKATA JAGANNADHA RAO, Dallas, TX (US)

(72) Inventor: ANIRUDHA SURABHI VENKATA JAGANNADHA RAO, Dallas, TX (US)

(21) Appl. No.: 18/524,210

(22) Filed: Nov. 30, 2023

Related U.S. Application Data
(60) Provisional application No. 63/429,151, filed on Dec. 1, 2022.

(51) Int. Cl. B62J 50/21; A61B 5/00; A61B 5/11; B62J 6/24; B62J 45/412; B62J 45/414

(52) U.S. Cl. CPC B62J 50/21; A61B 5/11; A61B 5/6893; B62J 6/24; B62J 45/412; B62J 45/414; A61B 2503/22; A61B 2560/0223; A61B 2562/0219

(57) ABSTRACT

A system for driving monitoring and analyzing and generating alerts to users in real-time, comprising, a driving assistance device connected to a first computing device and a second computing device over a network. The driving assistance device is configured to provide navigation guidance instructions to the user and monitor the orientation of an object when turns curvature using a plurality of sensors. The driving assistance device configured to send the orientation data of an object to a first computing device, where the first computing device configured to analyze the received orientation of the object data and calculating a lean angle of the object, and identify the difference between the calculated lean angle of the object and actual lean angle of the object when the user on route, thereby the driving assistance device alerts the users through audio, haptic feedback and/or visual signalling including LED lights indication, indications in mobile application etc.

### CROSS-REFERENCE TO RELATED APPLICATIONS

This patent application claims priority benefit of U.S. Provisional Patent Application No. 63/429,151, entitled “SYSTEM AND METHOD FOR DRIVING MONITORING AND ANALYZING AND GENERATING ALERTS TO USERS IN REAL-TIME”, filed on 1 Dec. 2022. The entire contents of the patent application is hereby incorporated by reference herein in its entirety.

### COPYRIGHT AND TRADEMARK NOTICE

This application includes material which is subject or may be subject to copyright and/or trademark protection. The copyright and trademark owner(s) has no objection to the facsimile reproduction by any of the patent disclosure, as it appears in the Patent and Trademark Office files or records, but otherwise reserves all copyright and trademark rights whatsoever.

### TECHNICAL FIELD

The disclosed subject matter relates generally to predicting user events. More particularly, the present disclosure relates to system and method for driving monitoring and analyzing and generating alerts to users in real-time.

### BACKGROUND

The automobile plays a vital role in the modern developing world. Therefore, the utilization of roadways is increasing daily, and this causes an increment in the rate of accidents due to unawareness of the road conditions while driving vehicles by the people/riders/drivers. Therefore, the accident rate is increasing daily, so it is necessary to take some preventive measures to avoid unwanted accidents. In comparison to an automobile, one is exposed to considerably higher risk in road traffic as a motor-bike rider like, for example, a motorcycle rider and/or a motor-trike rider and/or a quad rider and/or a scooter rider and/or a moped rider. Furthermore, riders of these two-wheeler vehicles are exposed to a higher degree of risk of injury when compared to other vehicles. Among other things, this is because of the different driving physics and the constantly unstable state of balance, as well as the particular physical and psychological stress when riding a motor bike and the limited field of view.

Especially, Motorcycle riding has grown in popularity in all countries. Motorcycles are maneuverable vehicles that provide riders with a sense of freedom and intense experience, especially perceived during cornering and accelerations. As motorcyclists get more experienced and improve their riding skills, they may become more self-confident and usually tend to push their limits seeking fun and excitement. This self-confidence on their riding technique and ability to control their motorcycles encourages most riders to underrate the risks when riding at very high speeds (e.g., more than double the speed limit). Being in this state of enthusiastic riding style may cause some riders to become overconfident in their abilities, resulting in a discrepancy between their perceived and actual limits. Therefore, in many cases, motorcyclists face motorcycle accidents/crashes. Most of the accidents/crashes are caused by riders' errors due to unawareness of the road conditions. The majority of accidents/crashes scenarios are the motorcycle going out of a curve at a relatively high speed, slide-out and falling due to over-braking, running wide of a curve/turn due to excess or inappropriate speed, or under-cornering, events that are often associated with limited visibility. Although inappropriate speed for the curve is the major cause in many cases. In addition the risks described above, riders of two wheelers are subject to additional risk of being hit by other drivers, due to limited visibility.

In the light of the aforementioned discussion, there exists a need for a system with novel methodologies that would overcome the above-mentioned challenges.

### SUMMARY

The following presents a simplified summary of the disclosure in order to provide a basic understanding of the reader. This summary is not an extensive overview of the disclosure and it does not identify key/critical elements of the invention or delineate the scope of the invention. Its sole purpose is to present some concepts disclosed herein in a simplified form as a prelude to the more detailed description that is presented later.

Exemplary embodiments of the present disclosure are directed towards a system and method for driving monitoring and analyzing and generating alerts to users in real-time.

An objective of the present disclosure is towards predicting and analyzing the turn of direction of object/subject.

Another objective of the present disclosure is towards providing analysis of movement behavior of a user based on combination of data from the sensors.

Another objective of the present disclosure is towards predicting user events and generating alerts to users in real-time.

Another objective of the present disclosure is towards providing a method and system for activating and initiating an emergency distress protocol/signal highlighting the user's exact geographical location to emergency services, personal and public contacts.

Another objective of the present disclosure is towards providing a method and system for activating and initiating an emergency distress protocol/signal by which the smart helmet system automatically begins recording and keeping a log of all the data which it receives so that this data can later be used in case of an enquiry, thereby acting as a deterrent for potential offenders.

Another objective of the present disclosure is towards measuring one or more linear accelerations and angular accelerations at each point of direction of turn.

Another objective of the present disclosure is towards notifying direction of turn of the vehicle/object.

Another objective of the present disclosure is towards providing a gyroscope value of the vehicle/object

Another objective of the present disclosure is towards providing blind spot of the road notifications to users.

Another objective of the present disclosure is directed towards providing turn notification of the object to the first end user through audio, haptic feedback and/or visual signaling including light emitting diode indication, indication in mobile application etc. by vibration motors.

Another objective of the present disclosure is directed towards analyzing the data collected by the multiple sensors to get additional insights of the direction of turn of the vehicle/object.

### BRIEF DESCRIPTION OF THE DRAWINGS

FIG. 1 is a block diagram depicting a schematic representation of a system for driving monitoring, analyzing, and generating alerts to users in real-time, in accordance with one or more exemplary embodiments.

FIG. 2 is a block diagram depicting a driving assistance device shown in FIG. 1, in accordance with one or more exemplary embodiments.

FIG. 3 is a block diagram depicting a schematic representation of the driving monitoring and analyzing module 114 shown in FIG. 1, in accordance with one or more exemplary embodiments.

FIG. 4 is an example free-body diagram, in accordance with one or more exemplary embodiments.

FIG. 5a is an example cyclist or rider on a normal road, FIG. 5b is an example of the bending of a cyclist or rider in curved road, in accordance with one or more exemplary embodiments.

FIG. 6 is a flow chart depicting an exemplary method for calculating lean angle of the object and banking angle of the object, in accordance with one or more exemplary embodiments.

FIG. 7 is a flowchart depicting an exemplary method for detecting banking angle of the object and alerts the first end user, in accordance with one or more exemplary embodiments.

FIG. 8 is a block diagram illustrating the details of a digital processing system in which various aspects of the present disclosure are operative by execution of appropriate software instructions.

### DETAILED DESCRIPTION OF EXAMPLE EMBODIMENTS

It is to be understood that the present disclosure is not limited in its application to the details of construction and the arrangement of components set forth in the following description or illustrated in the drawings. The present disclosure is capable of other embodiments and of being practiced or of being carried out in various ways. Also, it is to be understood that the phraseology and terminology used herein is for the purpose of description and should not be regarded as limiting.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced items. Further, the use of terms “first”, “second”, and “third”, and so forth, herein do not denote any order, quantity, or importance, but rather are used to distinguish one element from another.

Referring to FIG. 1 is a block diagram 100 depicting a schematic representation of a system for driving monitoring and analyzing and generating alerts to users in real-time, in accordance with one or more exemplary embodiments. The system 100 includes a driving assistance device 102, a processing device 103, a first computing device 106, a second computing device 108, a central database 112 and a drive monitoring and analyzing module 114. The system 100 may include multiple driving assistance devices 102, multiple processing devices 103, and multiple computing devices 106, 108. The system 100 may link multiple first computing device 106 and the second computing device 108 into a single hub. The user events may include, but not limited to, non-accidental emergency events relating to the vehicle (e.g., a theft of the vehicle), or emergency events relating specifically to the occupant(s) of the vehicle (e.g., a medical impairment of an occupant of the vehicle, regular events in the course of rough activity, a kidnapping or assault of an occupant of the vehicle, etc.), accidental emergency events relating to vehicle or other transport crashes, fires, medical emergencies, or other threats to safety, movements and motion, injury, abnormalities, and so forth.

The objects may include, but not limited to, vehicles, car seats, wristbands, helmets, headbands, and so forth. The subject may be a first end-user. The first end user may include, but not limited to, a driver, an athlete, a motorist, a passenger, a vehicle owner, a vehicle user, an individual, a rider, other individual riders and so forth.

The driving assistance device 102 may be an inertial measurement unit. The driving assistance device 102 may be configured to detect and track an object's motion in three-dimensional space, and allows the first end users to interact with the first computing device 106 by tracking motion in free space and delivering these motions as input commands. The driving assistance device 102 may be integrated into a vehicle, steering wheel, dashboard, car seats (if the user does not require an image capturing unit), headbands, helmets, electronic devices, wristbands and so forth. The driving assistance device 102 may be configured to detect/sense the impact events, emergency events, leaning & turning events, physical motion & movement interrupts, impacts or anomalies occur to the objects/subjects. The driving assistance device 102 may be configured to activate the impact protocol (emergency protocol) to establish the communication with the first computing device 106 and the second computing device 108 through the drive monitoring and analyzing module 114 via the network 110. The network 110 may include but not limited to, an Internet of things (IOT network devices), an Ethernet, a wireless local area network (WLAN), or a wide area network (WAN), a Bluetooth low energy network, a ZigBee network, a WIFI communication network e.g., the wireless high speed internet, or a combination of networks, a cellular service such as a 4G (e.g., LTE, mobile WiMAX) or 5G cellular data service, a RFID module, a NFC module, wired cables, such as the world-wide-web based Internet, or other types of networks may include Transport Control Protocol/Internet Protocol (TCP/IP) or device addresses (e.g. network-based MAC addresses, or those provided in a proprietary networking protocol, such as Modbus TCP, or by using appropriate data feeds to obtain data from various web services, including retrieving XML data from an HTTP address, then traversing the XML for a particular node) and so forth without limiting the scope of the present disclosure. The drive monitoring and analyzing module 114 may be configured to establish the communication between the impact event monitoring device 102 and the first computing device 106 through the network 110.

According to an exemplary aspect, the driving assistance device 102 may be integrated into a helmet system comprising a control unit-PCB wirelessly connected to a computing device over a network, the computing device may be configured to enable a user to use different functionalities without having to remove a helmet and access the first computing device and the control unit-PCB is configured to detect crashes while wearing the helmet by the user and notify the crash detected information to the computing device over the network.

According to an exemplary aspect, a system using machine learning, and more particularly deep learning using Long Short Term Memory for the analysis of the movement behavior of a user based on a combination of data from the sensor on hardware along with the computing device.

The first computing device 106 and the second computing device 108 may be operatively coupled to each other through the network 110. The first and second computing devices 106 and 108 may include but not limited to, a computer workstation, an interactive kiosk, and a personal mobile computing device such as a digital assistant, a mobile phone, a laptop, and storage devices, backend servers hosting the database and other software, and so forth. The first computing device 106 may be operated by the first end user. The first end user may include but not limited to driver, rider. The second computing device 108 may be operated by the second end user. The second end user may include, but not limited to, medical professionals, a medical examiner(s), an emergency responder(s), an emergency authority medical practitioner(s), a doctor(s), a physician(s), transport industry authorities, vehicle industry authorities, insurance companies, a family member(s), a friend(s), a relative(s), a neighbour(s), an emergency service provider(s), and so forth.

Although the first and second computing devices 106, 108 are shown in FIG. 1, an embodiment of the system 100 may support any number of computing devices. Each computing device supported by the system 100 is realized as a computer-implemented or computer-based device having the hardware or firmware, software, and/or processing logic needed to carry out the intelligent messaging techniques and computer-implemented methodologies described in more detail herein.

The drive monitoring and analyzing module 114, which is accessed as mobile applications, web applications, software that offers the functionality of accessing mobile applications, and viewing/processing of interactive pages, for example, are implemented in the first and second computing devices 106, 108 as will be apparent to one skilled in the relevant arts by reading the disclosure provided herein. The drive monitoring and analyzing 114 may be downloaded from the cloud server (not shown). For example, drive monitoring module 114 may be any suitable applications downloaded from, GOOGLE PLAY® (for Google Android devices), Apple Inc.'s APP STORE® (for Apple devices, or any other suitable database). In some embodiments, the drive monitoring and analyzing module 114 may be software, firmware, or hardware that is integrated into the first and second computing devices 106, 108.

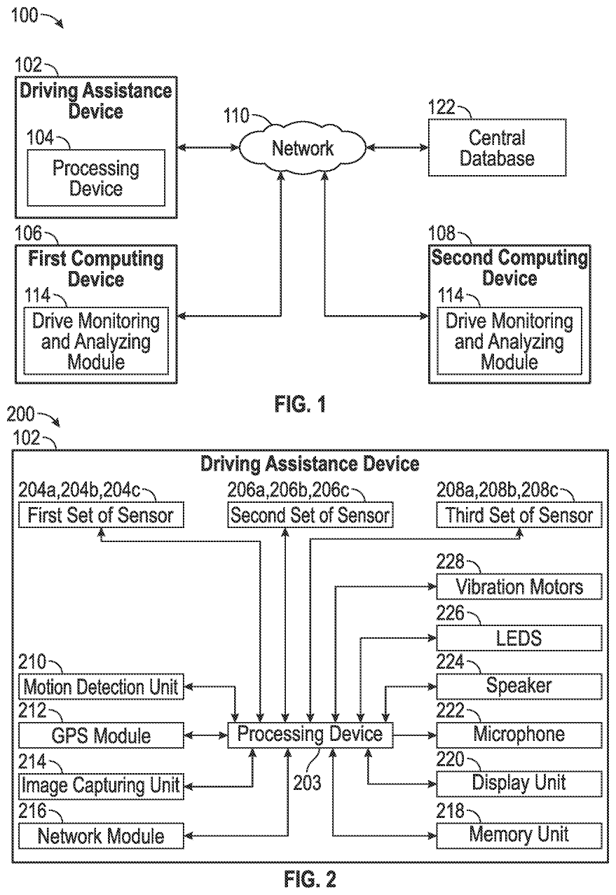

The processing device 104 may include, but not limited to, a microcontroller (for example ARM 7 or ARM 11), a raspberry pi3 or a Pine 64 or any other 64 bit processor which can run Linux OS, a microprocessor, a digital signal processor, a microcomputer, a field programmable gate array, a programmable logic device, a state machine or logic circuitry, Arduino board. A set of sensors (204a, 204b and 204c, 206a, 206b and 206c, 208a, 208b and 208c shown in FIG. 2) may be electrically coupled to the processing device 103.

Referring to FIG. 2 is a block diagram 200 depicting the user driving assistance device 102 shown in FIG. 1, in accordance with one or more exemplary embodiments. The driving assistance device 102 includes the processing device 203, a first set of sensors 204a, 204b, and 204c, a second set of sensors 206a, 206b and 206c, a third set of sensors 208a, 208b, and 208c, an motion detection unit 210, a GPS module 212, an image capturing unit 214, a network module 216, a memory unit 218, and a display unit 220, a microphone 222, two speakers 224, LED lights 226, vibration motors 228.

The first set of sensors 204a, 204b, 204c, the second set of sensors 206a, 206b and 206c, the third set of sensors 208a, 208b, and 208c may include, but not limited to, ultrasonic sensor, gyroscope sensor, accelerometers, direction sensor, speed sensor, compasses, pressure sensors, and magnetometers.

The first set of sensors 204a, 204b, and 204c may be electrically coupled to the processing device 203 and is configured to measure the linear acceleration and angular acceleration of an object/subject at each point. The second set of sensors 206a, 206b, and 206c may be electrically coupled to the processing device 203 and is configured to calibrate the exact orientations by measuring the Euler angles and/or quaternions. The third set of sensors 208a, 208b and 208c may be electrically coupled to the processing device 203 and is configured to monitor vital statistics, the rotational angle of the head of the individual or the object at the time of the impact event. The third set of sensors 208a, 208b and 208c may also be configured to provide additional data to the second end users to properly diagnose the extent and severity of the impact event. The speed sensor may be configured to detect the object's speed. The Object may include but not limited a car, bike, cycle. The ultrasonic sensor may be placed at blind spot detection angles and configured to scan and report if any object/vehicle is in the user's blind spot. The speed sensor may be configured to measure the speed of the vehicle. The vehicle may include but not limited to, bike, car and so forth.

The motion detecting unit 210 may be electrically coupled to the processing device 203 and is configured to measure changes in the orientations for having a continuous replication of the movement and/or motion of the objects/subjects. Based on the motion detection unit 210, identifying the location and direction of an impact on a surface.

The GPS module 212 may be electrically coupled to the processing device 104 and is configured to detect the accurate location of the impact events that occur to the objects/subjects. The image capturing unit 214 may be electrically coupled to the processing device 203 and is configured to record the video of the subjects/objects and capture the objects/subjects. For example, similar to live media, in the sense the image capturing unit 214 starts recording as soon as the first end user opens the drive monitoring and analyzing module 114 before the live media is captured. The live media may include, but not limited to, live photos, live media files, and so forth. The image capturing unit 214 may be configured to recreate the captured impact events (live media) in a 3D space. The network module 216 may be electrically coupled to the processing device 203 and is configured to connect the driving assistance device 102 with the first computing device 106. The network module 216 may be configured to send the turn signals as notifications to the second end user. The notifications include audio and haptic prompts, including but not limited to, SMS, alerts, email, warnings, and so forth. The network module 216 may also be configured to send a geographical location as a communication link and the information identifying the location of the objects\subjects to the second computing device 108 to communicate the portion of data stored in the memory unit 218. The information stored in memory unit 218 may be preserved at least until an acknowledgment of receipt is received representing successful transmission through the communication link. The memory unit 218 may be electrically coupled to the processing device 203 and is configured to receive movement or motion output and stores at least a portion of motion commencing at and/or before said determination. The display unit 220 may be electrically coupled to the processing device 203 and is configured to display the sensor data, impact notifications, and so forth. According to an exemplary embodiment of the present disclosure, the driving assistance device 102 may be connected to the microphone 222, the two speakers 224 with wireless communication.

The speakers 224 of the driving assistance device 102 may be configured to send audio when uses/rider make turn the vehicle/object. The light emitting diodes (LEDs) 226 may be configured to alert users regarding user events including circumstances of user events.

The driving assistance device 102 may be configured to send immediate sensory feedback to the user through light-emitting diodes (LEDs) 226 and vibration motors 228. The vibrations may be haptic feedback, this haptic feedback may be generated with the help of vibration motors 228. This enables the user to navigate the lanes on the road without having to take their eyes off the road.

The first set of sensors 204a, 204b, 204c, the second set of sensors 206a, 206b, and 206c and the third set of sensors 208a, 208b, and 208c may be configured to calculate the Pitch, Roll and Yaw data from the impact event. Pitch, Roll, and Yaw are the respective rotations of the object and/or the subject around the X, Y and Z axes respectively. The first set of sensors 204a, 204b, 204c, the second set of sensors 206a, 206b, 206c and third set of sensors 208a, 208 and 208c may include but not limited to IMU (Inertial measurement unit) sensor. The processing device 203 may be configured to obtain the values of Ax, Ay, Az, GYx, GYy and GYZ.

Pitch=180*atan 2(Ax,Sqrt((Ay*Ay)+(Az*Az))/pi

Roll=180*atan 2(Ay,Sqrt((Ax*Ax)+(Az*Az))/pi

Yaw=180*atan 2(Az,Sqrt((Ax*Ax)+(Az*Az))/pi

Yaw value may change and is not the absolute value of it. Therefore, considering an assumed value initially, preferably 0. In order to calculate yaw, first find the up direction using the accelerometer to measure g (gravity) (opposite to g is upwards) and using the gyroscope to measure the rate of turn on each axis. Scale these by the correct amount based on the orientation to obtain a yaw rate. Thereafter, integrating this yaw rate over time to obtain yaw values.

Tracking things while under significant motion is complex. Therefore, a pre-tuned KALMAN filter may be used to obtain accurate data. There is a buffer time of around 30-40 seconds after the impact event before initializing the analysis.

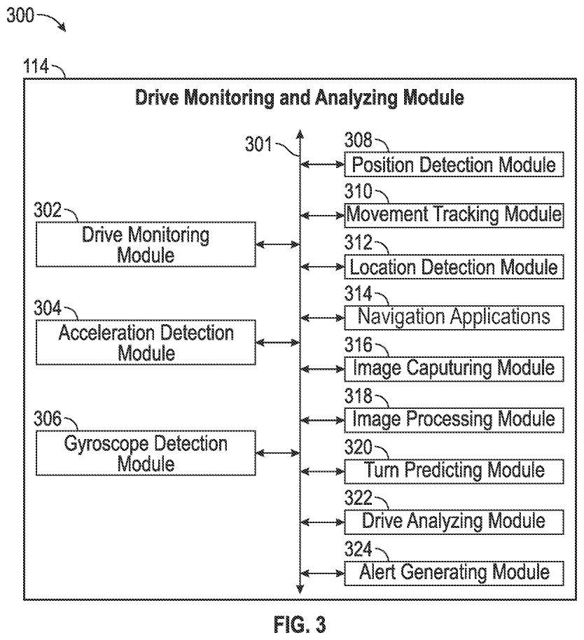

Referring to FIG. 3 is a block diagram 300 depicting a schematic representation of the drive monitoring and analyzing module 114 shown in FIG. 1, in accordance with one or more exemplary embodiments. The drive monitoring and analyzing module 114 includes a bus 301, a drive monitoring module 302, an acceleration detection module 304, a gyroscope detection module 306, a position detection module 308, a movement tracking module 310, a location detection module 312, a navigation applications 314, an image capturing module 316, an image processing module 318, a turn predicting module 320, a drive analyzing module 322, an alert generating module 324. The bus 301 may include a path that permits communication among the modules of the drive monitoring and analyzing module 114 installed on the first computing device and computing device 106, 108. The term “module” is used broadly herein and refers generally to a program resident in the memory of the first computing device 106, and the second computing device 108. The drive monitoring and analyzing module 114 may include machine learning techniques, logical algorithms, double authentication & validation algorithms and computer-implemented pattern recognition techniques to detect anomalies or variations in normal behavior.

The driving monitoring module 302 may be configured to read the sensor data of the objects/subjects and stores in the central database 112 or onboard memory component or transfer it to an another computer. The sensor data may be measured by the driving assistance device 102. The sensor data may include, but not limited to, quaternions, Euler angles, vital statistics, the rotational angle of the head of the individual or the object, movement and/or motion of the individual or the object, geo location, acceleration and gyroscope vectors, velocity, location and so forth. The acceleration detection module 304 may be configured to sense the acceleration information of the subject/object. The acceleration detection module 304 may be configured to obtain acceleration information relative to the sensor. The acceleration detection module 304 may include one or more acceleration sensors, an accelerometer and the like. The gyroscope detection module 306 may be configured to measure and maintain the orientation and angular velocity of a subject/object.

The position detection module 308 may be configured to fetch the object/subject positions (geo-location) “x” seconds before the object turns on the road and “x” seconds after the object turns on the road. The movement tracking module 310 may be configured to track the head movements with yaw, pitch and roll data. The location detection module 312 may be configured to provide accurate location of the turning of the road. The driving monitoring and analyzing module 114 may be associated with the google maps API (application programming interface) 314 configured to provide navigation guidance instructions. The first end user may get turn-by-turn voice guided instructions on how to at arrive at a given destination through the google maps API (application programming interface) 314. The first end user may include but not limited to the driver, rider, cyclist and so forth. The image capturing module 316 may be configured to capture the objects/subjects. The image capturing module 316 may be configured to move the media files of the bending of cyclist/biker in a curved road with respect time to time. The media files may include but not limited to, image, pictures, videos, GIF's and so forth. The media files may be moved front and back with respect to time and the object/subject may be portrayed accordingly. The image processing module 318 may be configured to convert the resulting “2x” seconds of the object/subject positions into a short animation/video by which the accurate object/subject positions at the time of the turn may be reproduced.

The turn predicting module 320 may be configured to predict the lean angle of the cyclist on the road based on the received information includes the gyroscope, speed, acceleration of the first end user. The first end user may be a cyclist or rider or driver of other two-wheeler or three vehicles. The turn predicting module 320 may configured to transmit predict the lean angle of the cyclist on the road. The drive analyzing module 322 may receive information from the acceleration detection module, gyroscope detection module, image processing module and analyze received information. The drive analyzing module 322 may be configured to calculate the rate of change of deceleration(jerk), lean angle, and rate of change of angular displacement. The drive analyzing module 322 may be configured to analyze the yaw, pitch and roll data with machine learning techniques and the deep learning techniques to determine the banking angle and radius of curvature of the road or track. The drive analyzing module 322 may be configured to identify the difference between the predict lean angle of the cyclist on road and calculated lean angle of the cyclist on road. The alert generating module 324 may be configured to generate the notifications of direction of turn of the first end user to the second computing device. The alert generating module 324 may also be configured to generate alerts to the first end user through audio prompts, haptic feedback, visual prompts.

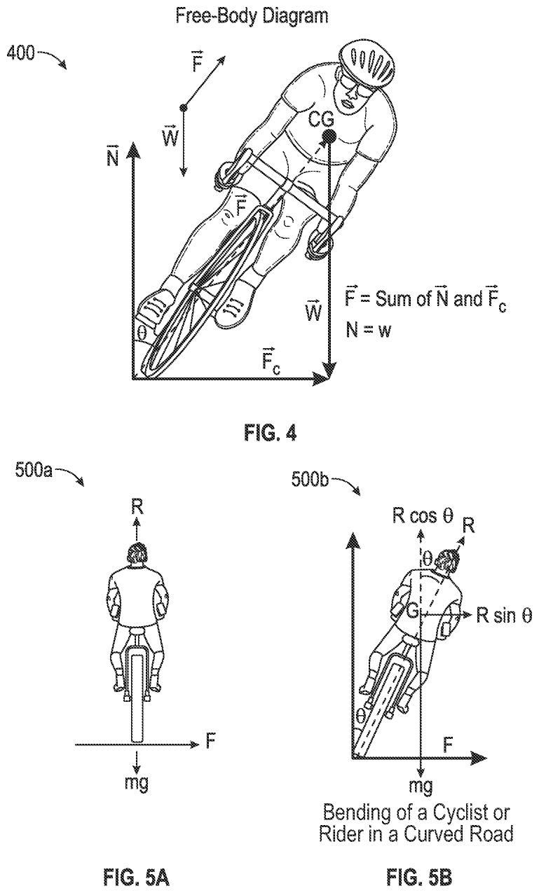

Referring to FIG. 4 is an exemplary free-body diagram 400 and FIG. 5a, 5b diagrams 500a, 500b, in accordance with one or more exemplary embodiments. The first end user or rider may make to left or right turn, a lean angle θ is made by the rider with respect to the ground surface. The frictional force between the track's surface and the rider's tire of the cycle or bike and creates the centripetal force that the rider feels when moving down a curved track. Then the normal force makes the same angle θ with the vertical. When the car takes a turn, there are two forces acting on the car: a) Gravitational force mg (downwards) b) Normal force N (perpendicular to surface). It can be resolved into two components. N cos θ and N sin θ. The component N cos θ balances the downward gravitational force ‘mg’ and component N sin θ will provide the necessary centripetal acceleration. By using Newton's second law
N cos θ=mg
N sin θ=mv²/r
By dividing the equations we get
tan θ<=v²/rg
where r—radius of curvature
   g—gravitational constant
      The safe speed of the rider during the turn is determined by the banking angle and radius of curvature of the road or track. The cycle or the bike begins to slide outward if the speed is higher than this safe speed, but frictional force kicks in and adds more centripetal force to stop the outward skidding. Additionally, if the cycle or the bike is moving at a speed that is just a little bit too fast, it begins to skid inward. This causes frictional force, which lowers the centripetal force and prevents inward skidding. However, frictional force cannot prevent the automobile from sliding if the speed of the vehicle is considerably higher than the appropriate speed. This equation supports the system in understanding whether a proper turn is being taken by the user/rider satisfying the above condition.

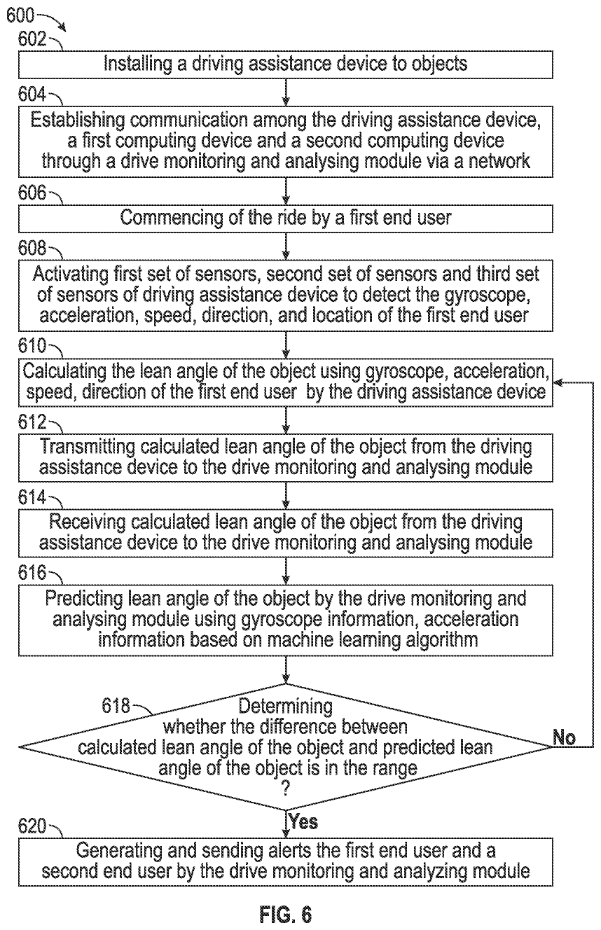

Referring to FIG. 6 is a flow chart 600 depicting an exemplary method for calculating the lean angle of the object and banking angle of the object, in accordance with one or more exemplary embodiments. As an option, the exemplary method 600 is carried out in the context of the details of FIG. 1, FIG. 2, FIG. 3, FIG. 4, and FIG. 5. However, the exemplary method 600 is carried out in any desired environment. Further, the aforementioned definitions are equally applied to the description below.

The method commences at step 602, installing a driving assistance device to objects. Thereafter at step 604, establishing communication among the driving assistance device, the first computing device and the second computing device through the drive monitoring and analyzing module via the network. Thereafter at step 606, commencing of the ride by the first end user. Thereafter at step 608, activating the first set of sensors, the second set of sensors and the third set of sensors of the driving assistance device to detect the gyroscope, acceleration, speed, direction, and location of the first end user. Thereafter at step 610, calculating the lean angle of the object using gyroscope, acceleration, speed, and direction of the first end user by the driving assistance device. Thereafter at step 612, transmitting calculated the lean angle of the object from the driving assistance device to the drive monitoring and analyzing module. Thereafter at step 614, receiving calculated lean angle of the object from the driving assistance device to the drive monitoring and analyzing module. Thereafter at step 616, predicting the lean angle of the object by the drive monitoring and analyzing module using gyroscope information, and acceleration information based on machine learning algorithm. Determining whether the difference between calculated lean angle of the object and predicted lean angle of the object is in the range, at step 618. If the answer at step 618 is Yes, the method continuous at step 620, generating and sending alerts the first end user and a second end user by the drive monitoring and analyzing module. If the answer at step 618 is No, the method goes to the step 610.

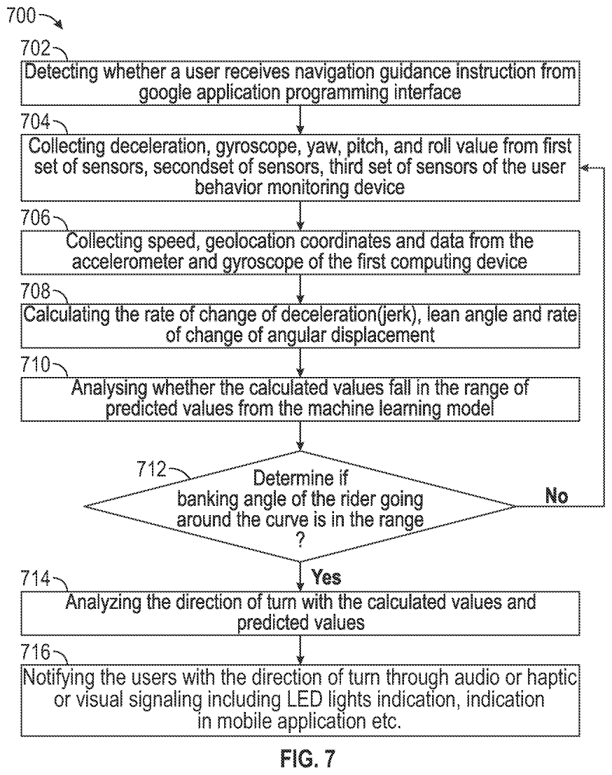

Referring to FIG. 7 is a flowchart 700 depicting an exemplary method for detecting banking angle of the object and alerts the first end user, in accordance with one or more exemplary embodiments. As an option, the exemplary method 700 is carried out in the context of the details of FIG. 1, FIG. 2, FIG. 3, FIG. 4, FIG. 5 and FIG. 6. However, the exemplary method 700 is carried out in any desired environment. Further, the aforementioned definitions are equally applied to the description below.

The method commences at step 702, detecting whether a first end user receives navigation guidance instruction from google application programming interface. Thereafter at step 704, collecting deceleration, gyroscope, yaw, pitch, and roll value from first set of sensors, second set of sensors, third set of sensors of the driving assistance device. Thereafter at step 706, collecting speed, geolocation coordinates and data from the accelerometer and gyroscope of the first computing device. Thereafter at step 708, calculating the rate of change of deceleration(jerk), lean angle and rate of change of angular displacement. Thereafter at step 710, analysing whether the calculated values fall in the range of the predicted values from the machine learning model. Thereafter at step 712, determine if banking angle of the rider going around the curve is in the range. If the answer at step 712 is Yes, the method continuous at step 714, analyzing the direction of turn with the calculated values and predicted values. Thereafter at step 716, notifying the users with the direction of turn through audio or haptic or visual signaling including LED lights indication, indication in mobile application etc. If the answer at step 712 is No, method goes to step 704.

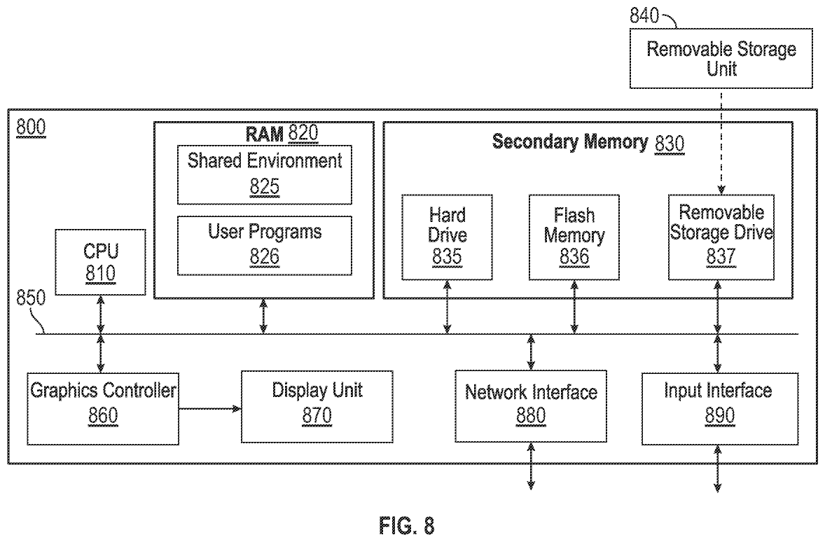

Referring to FIG. 8 is a block diagram 800 illustrating the details of a digital processing system 800 in which various aspects of the present disclosure are operative by execution of appropriate software instructions. The Digital processing system 800 may correspond to the computing devices 106, 108 (or any other system in which the various features disclosed above can be implemented).

Digital processing system 800 may contain one or more processors such as a central processing unit (CPU) 810, random access memory (RAM) 820, secondary memory 830, graphics controller 860, display unit 870, network interface 880, and input interface 890. All the components except display unit 870 may communicate with each other over communication path 850, which may contain several buses as is well known in the relevant arts. The components of FIG. 8 are described below in further detail.

CPU 810 may execute instructions stored in RAM 820 to provide several features of the present disclosure. CPU 810 may contain multiple processing units, with each processing unit potentially being designed for a specific task. Alternatively, CPU 810 may contain only a single general-purpose processing unit.

RAM 820 may receive instructions from secondary memory 830 using communication path 850. RAM 820 is shown currently containing software instructions, such as those used in threads and stacks, constituting shared environment 825 and/or user programs 826. Shared environment 825 includes operating systems, device drivers, virtual machines, etc., which provide a (common) run time environment for execution of user programs 826.

Graphics controller 860 generates display signals (e.g., in RGB format) to display unit 870 based on data/instructions received from CPU 810. Display unit 870 contains a display screen to display the images defined by the display signals. Input interface 890 may correspond to a keyboard and a pointing device (e.g., touch-pad, mouse) and may be used to provide inputs. Network interface 880 provides connectivity to a network (e.g., using Internet Protocol), and may be used to communicate with other systems (such as those shown in FIG. 1) connected to the network 110.

Secondary memory 830 may contain hard drive 835, flash memory 836, and removable storage drive 837. Secondary memory 830 may store the data software instructions (e.g., for performing the actions noted above with respect to the Figures), which enable digital processing system 800 to provide several features in accordance with the present disclosure.

Some or all of the data and instructions may be provided on removable storage unit 840, and the data and instructions may be read and provided by removable storage drive 837 to CPU 810. Floppy drive, magnetic tape drive, CD-ROM drive, DVD Drive, Flash memory, removable memory chip (PCMCIA Card, EEPROM) are examples of such removable storage drive 837.

Removable storage unit 840 may be implemented using medium and storage format compatible with removable storage drive 837 such that removable storage drive 837 can read the data and instructions. Thus, removable storage unit 840 includes a computer readable (storage) medium having stored therein computer software and/or data. However, the computer (or machine, in general) readable medium can be in other forms (e.g., non-removable, random access, etc.).

In this document, the term “computer program product” is used to generally refer to removable storage unit 840 or hard disk installed in hard drive 835. These computer program products are means for providing software to digital processing system 800. CPU 810 may retrieve the software instructions, and execute the instructions to provide various features of the present disclosure described above.

The term “storage media/medium” as used herein refers to any non-transitory media that store data and/or instructions that cause a machine to operate in a specific fashion. Such storage media may comprise non-volatile media and/or volatile media. Non-volatile media includes, for example, optical disks, magnetic disks, or solid-state drives, such as storage memory 830. Volatile media includes dynamic memory, such as RAM 820. Common forms of storage media include, for example, a floppy disk, a flexible disk, hard disk, solid-state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge.

Storage media is distinct from but may be used in conjunction with transmission media. Transmission media participates in transferring information between storage media. For example, transmission media includes coaxial cables, copper wire and fiber optics, including the wires that comprise bus (communication path) 850. Transmission media can also take the form of acoustic or light waves, such as those generated during radio-wave and infra-red data communications.

Reference throughout this specification to “one embodiment”, “an embodiment”, or similar language means that a particular feature, structure, or characteristic described in connection with the embodiment is included in at least one embodiment of the present disclosure. Thus, appearances of the phrases “in one embodiment”, “in an embodiment” and similar language throughout this specification may, but do not necessarily, all refer to the same embodiment.

Furthermore, the described features, structures, or characteristics of the disclosure may be combined in any suitable manner in one or more embodiments. In the above description, numerous specific details are provided such as examples of programming, software modules, user selections, network transactions, database queries, database structures, hardware modules, hardware circuits, hardware chips, etc., to provide a thorough understanding of embodiments of the disclosure.

Although the present disclosure has been described in terms of certain preferred embodiments and illustrations thereof, other embodiments and modifications to preferred embodiments may be possible that are within the principles of the invention. The above descriptions and figures are therefore to be regarded as illustrative and not restrictive.

Thus the scope of the present disclosure is defined by the appended claims and includes both combinations and sub-combinations of the various features described hereinabove as well as variations and modifications thereof, which would occur to persons skilled in the art upon reading the foregoing description.

What is claimed is:

1. A system for driving monitoring, analyzing, and generating alerts to users in real-time, comprising:
a driving assistance device integrated into at least one of a vehicle, a vehicle peripheral components or to a rider accessories, the driving assistance device comprising a first set of sensors, a second set of sensors, and a third set of sensors electrically connected to a processing device, wherein the first set of sensors are configured to measure linear acceleration and angular acceleration of an object, the second set of sensors are configured to calibrate orientations of the object, the third set of sensors are configured to monitor vital parameters of the rider and a rotational angle of the rider's head;
a drive monitoring and analyzing module incorporated in a first computing device and a second computing device, wherein the first computing device and the second computing device operatively coupled to each other through a network, whereby the drive monitoring and analyzing module configured to facilitate communication between the driving assistance device and the first computing device and the second computing device via the network such as Bluetooth, Wi-Fi, or internet, and the drive monitoring and analyzing module reads the sensor data received from the driving assistance device and stores the sensor data in a central database or onboard memory component or transfer it to another computing device;
the drive monitoring and analyzing module comprising machine learning techniques, logical algorithms, double authentication and validation algorithms, and computer implemented pattern recognition techniques for analyzing the sensor data received from the driving assistance device, thereby generating alerts in real-time.

2. The system of claim 1, wherein the first set of sensors comprises at least one of an accelerometer, a gyroscope, a direction sensor, or a speed sensor.

3. The system of claim 1, wherein the second set of sensors comprises at least one of an ultrasonic sensor or a magnetometer or any other orientation measurement sensor.

4. The system of claim 1, wherein the third set of sensors comprises at least one of a heart rate monitor, a head rotation sensor, or a temperature sensor or any other physical parameter sending component.

5. The system of claim 1, wherein the drive monitoring and analyzing module is configured to detect events selected from the group consisting of impact events, emergency events, leaning and turning events, physical motion and movement interrupts, impacts, and anomalies occurring to the object or rider.

6. The system of claim 1, wherein the drive monitoring and analyzing module comprises a drive monitoring module configured to read the sensor data and store it in the central database or onboard memory component or transfer it to an another computing device.

7. The system of claim 1, wherein the drive monitoring and analyzing module comprises an acceleration detection module configured to sense the acceleration information of the object.

8. The system of claim 1, wherein the drive monitoring and analyzing module comprises a gyroscope detection module configured to measure and maintain the orientation and angular velocity of the object.

9. The system of claim 1, wherein the drive monitoring and analyzing module comprises a position detection module configured to fetch the object's orientation coordinates.

10. The system of claim 1, wherein the drive monitoring and analyzing module comprises a movement tracking module configured to track rider head movements with yaw, pitch, and roll data.

11. The system of claim 1, wherein the drive monitoring and analyzing module comprises a location detection module configured to provide accurate geolocation information for determining turns in the road.

12. The system of claim 1, wherein the drive monitoring and analyzing module comprises an image capturing module and an image processing module.

13. The system of claim 1, wherein the drive monitoring and analyzing module comprises a turn predicting module configured to predict a lean angle of the rider on the road based on received information, including data from the gyroscope, speed, and acceleration of the rider.

14. The system of claim 1, wherein the drive monitoring and analyzing module comprises an alert generating module configured to generate notifications or alerts indicating the direction of turn of the object or rider to the second computing device through audio, haptic feedback, visual signaling including light-emitting diode indications and indications within a mobile application.

15. The system of claim 1, wherein the drive monitoring and analyzing module comprises an alert generating module configured to generate notifications or alerts indicating the direction of turn to the rider through audio, haptic feedback, visual signaling including light-emitting diode indications and indications within a mobile application.

16. The system of claim 15, wherein the haptic feedback is generated by vibration motors, thereby enabling the rider to navigate lanes on the road without having to take their eyes off the road.

17. The system of claim 1, wherein the drive monitoring and analyzing module comprises an alert generating module configured to generate notifications or alerts indicating the direction of turn of the object or rider to other individual riders through audio, haptic feedback, visual signaling including light-emitting diode indications, and indications within a mobile application.

18. The system of claim 1, wherein the drive monitoring and analyzing module comprises a drive analyzing module configured to receive information from the acceleration detection module, the gyroscope detection module, and the image processing module, alert generating module and analyze the information to calculate the rate of change of deceleration (jerk), lean angle, and rate of change of angular displacement.

19. A method for driving monitoring, analyzing, and generating alerts to users in real-time, comprising:
installing a driving assistance device to a vehicle, a vehicle peripheral components or to a rider accessories;
establishing communication among the driving assistance device, a first computing device, and a second computing device through a drive monitoring and analyzing module via a network;
activating multiple sensors of the driving assistance device to detect gyroscope, acceleration, speed, direction, and location of the rider;
calculating lean angle of an object using gyroscope, acceleration, speed, and direction of the rider by the driving assistance device;
transmitting the calculated lean angle of the object from the driving assistance device to the drive monitoring and analyzing module;
predicting the lean angle of the object by the drive monitoring and analyzing module using gyroscope information and acceleration information based on a machine learning algorithm;
determining whether the difference between the calculated lean angle of the object and the predicted lean angle of the object is in a predetermined range; and
generating and sending alerts to the rider and an emergency authority by the drive monitoring and analyzing module.

20. The method of claim 19, further comprising a step of calibrating a first set of sensors, a second set of sensors, and a third set of sensors incorporated in the driving assistance device.
